(19) United States
(12) Patent Application Publication
(10) Pub. No.: US 2005/0186271 A1
(43) Pub. Date: Aug. 25, 2005

(54) Process for dispersing a fluid in a mass of solid particles

(76) Inventors: Paul J. Sheskey, Midland, MI (US); Colin M. Keary, Midland, MI (US)

Correspondence Address: THE DOW CHEMICAL COMPANY;INTELLECTUAL PROPERTY SECTION, P. O. BOX 1967, MIDLAND, MI, 48641-1967, US

(21) Appl. No.: 10/785,327

(22) Filed: Feb. 24, 2004

(51) Int. Cl. A61K 9/20

(52) U.S. Cl. 424/464; 264/109

(57) ABSTRACT

A fluid is dispersed in a mass of solid particles in a process wherein a) a gas is contacted with a fluid composition comprising i) from 0.001 to 30 weight percent of a surfactant having a weight average molecular weight of up to 30000 and ii) from 99.999 to 70 weight percent of a liquid diluent, based on the total weight of the surfactant i) and the liquid diluent ii), and b) the produced foam is contacted with solid particles of an average size of less than 2500 micrometers, wherein a therapeutic agent is comprised in the fluid composition or in the mass of solid particles or both.

### BACKGROUND OF THE INVENTION

The present invention relates to a process for dispersing a fluid in a mass of solid particles, such as a powder, and to a process for preparing tablets.

Processes for dispersing a fluid in solid particles have been known for a long time. Such processes are typically used for coating the solid particles with components comprised in the fluid or for granulating powders.

Granulation is widely used in the pharmaceutical industry as a particle size enlargement process. Benefits of granulation include improved flowability of the dried granulated particles, the elimination of hazardous dusts and significant reductions in the volume of material that must be subsequently handled. According to one well-known method a binder is dissolved in a liquid and applied to a powder mass. According to another method a dry binder is mixed with a powder to be granulated and a liquid such as water is added, which activates the binder. Unfortunately, in these processes there is the danger of inhomogeneous distribution of the liquid in the powder. Spots of powders with too much liquid result in the formation of large lumps whereas spots of powders with an insufficient amount of liquid do not properly form agglomerates. In American Pharmaceutical Review, Volume 3, Issue 4, Winter 2000, pages 33-36, “Granulation and Scale-Up Issues in Solid Dosage Form Development”, Tony Hlinak explains that fundamental models allowing the prediction of granulation times from measurements of ingredient and binding solutions properties do not exist, forcing the industry toward trial and error approaches to developing the granulation process.

One way of solving the above-mentioned problems of granulating powders is disclosed in the Patent Application WO 03/020244, which has been published on Mar. 13, 2003 and which has been invented by the same inventors as the process of the present patent application. It discloses a process for dispersing a fluid in solid particles wherein a) a gas is contacted with a fluid composition which comprises i) from 0.01 to 30 weight percent of a polymer and ii) from 99.99 to 70 weight percent of a liquid diluent, based on the total weight of the polymer i) and the liquid diluent ii), to produce a foam. The produced foam is contacted with solid particles of an average size of less than 2500 micrometers. The weight ratio between the foam and the solid particles is from 1:20 to 1:0.2. The polymer i) which is used as a foaming agent generally has a weight average molecular weight of at least 10,000. Preferred polymers are cellulose ethers and cellulose esters. The process taught in WO 03/020244 provides a highly improved granulation process.

However, in view of the wide use and high commercial importance of granulation processes in the pharmaceutical industry, it would still be desirable to find new granulation processes to enrich the art.

### SUMMARY OF THE INVENTION

One aspect of the present invention is a process for dispersing a fluid in a mass of solid particles which comprises the steps of
a) contacting a gas with a fluid composition comprising i) from 0.001 to 30 weight percent of a surfactant having a weight average molecular weight of up to 30000 and ii) from 99.999 to 70 weight percent of a liquid diluent, based on the total weight of the surfactant i) and the liquid diluent ii), and
b) contacting the produced foam with solid particles of an average size of up to 2500 micrometers,
wherein a therapeutic agent is comprised in the fluid composition or in the mass of solid particles or both.

Another aspect of the present invention is a granular material which is producible according to the above-mentioned process by contacting the foam with the solid particles and agglomerating the particles.

Another aspect of the present invention is a process for preparing tablets wherein step b) in the above-mentioned process comprises contacting the foam with the solid particles and agglomerating the particles to produce a granular material and the granular material is pressed to tablets.

### SHORT DESCRIPTION OF THE FIGURES

FIG. 1 illustrates drug dissolution from tablets prepared according to the process of the present invention containing the model drug Diphenhydramine HCl.

FIG. 2 represents drug dissolution from tablets prepared according to the process of the present invention containing the model drug hydrochlorothiazide.

### DETAILED DESCRIPTION OF THE INVENTION

The present invention relates to a process for dispersing a fluid in a mass of solid particles which comprises the steps a) and b) below. The term as used herein includes various ways of dispersion. For example, the fluid can stay at the surface of the individual solid particles without penetration into the individual solid particles; or the fluid can partially penetrate into the solid particles or can penetrate into some of the particles and remain at the surface of other particles; or the solid particles can absorb the fluid such that the foam penetrates into the solid particles.

As a starting material for step a) of the process of the present invention a fluid composition is prepared which comprises i) at least 0.001 percent, preferably at least 0.005 percent, more preferably at least 0.01 percent and most preferably at least 0.05 percent of a surfactant, and ii) up to 99.999 percent, preferably up to 99.995, more preferably up to 99.99 percent and most preferably up to 99.95 percent of a liquid diluent ii), based on the total weight of the polymer i) and the liquid diluent ii). The fluid composition comprises i) up to 30 percent, preferably up to 15 percent, more preferably up to 10 percent, and most preferably up to 5 percent of a surfactant, and ii) at least 70 percent, preferably at least 85 percent, more preferably at least 90 percent, and most preferably at least 95 percent of a liquid diluent ii), based on the total weight of the polymer i) and the liquid diluent ii).

The fluid composition may comprise one or more different surfactants, but their total amount should be within the range stated above.

The surfactant used in the present invention has a weight-average molecular weight Mw of up to 30000, preferably up to 9000, more preferably up to 5000, even more preferably up to 2000. The most preferred surfactants are non-polymeric compounds with a molecular weight of up to 1000, preferably up to 700. The weight average molecular weight can be determined by light scattering according to the Standard Test Method ASTM D-4001-93 (1999).

Surfactants which are useful in the process of the present invention are generally compounds with a hydrophilic head and a hydrophobic end. Anionic, cationic, amphoteric and nonionic surfactants are useful. In many cases nonionic surfactants are preferred over anionic, cationic or amphoteric surfactants.

As anionic surfactants preferably one or more substances from the group of carboxylic acids, carboxylic half-esters, sulfonic acids, preferably from the group of fatty acids, fatty alkylsulfuric acids and alkylarylsulfonic acids; sulfuric acid half-esters of long chain alcohols, alkylethersulfonic acids, like the alkylsulfuric acids; alkanesulfonic acids, or olefinsulfonic acids may be used. Alkali metal salts, preferably the sodium or potassium salts; or ammonium salts of the listed acids are also useful. Accordingly, an alkali metal salt, particularly the sodium salt, is also meant each time in the present description a free acid is mentioned. To achieve adequate surface-active properties, the compounds should have long-chain hydrocarbon radicals, thus have at least 6 carbon atoms in the alkyl or alkenyl radical. Usually the carbon chains in the anionic surfactants contain 6 to 40, preferably 8 to 30, and more preferably 12 to 22 carbon atoms. Preferred carboxylic acids are hexanoic acid (caproic acid), heptanoic acid (enanthic acid), octanoic acid (caprylic acid), nonanoic acid (pelargonic acid), decanoic acid (capric acid), or undecanoic acid. More preferably fatty acids are used, such as dodecanoic acid (lauric acid), tetradecanoic acid (myristic acid), hexadecanoic acid (palmitic acid), octadecanoic acid (stearic acid), eicosanoic acid (arachic acid), docosanoic acid (behenic acid), tetraconsanoic acid (lignoceric acid), hexacosanoic acid (cerotic acid), triacotanoic acid (melissic acid), and the unsaturated species 9c-hexadecenoic acid (palmitoleic acid), 6c-octadecenoic acid (petroselic acid), 6t-octadecenoic acid (petroselaidic acid), 9c-octadecenoic acid (olaic acid), 9t-octadecenoic acid (elaidic acid), 9c,12c-octadecadienoic acid (linoleic acid), 9t,12t-octadecadienoic acid (linolaidic acid), and 9c,12,15c-octadecatrienoic acid (linolenic acid). Also mixtures of fatty acids are useful, such as coconut oil fatty acid, palm kernel oil fatty acid, tallow fatty acid, hardened tallow fatty acids, palmitic/stearic acid mixtures and soybean oil fatty acid.

Alkylphosphates, sulfuric acid half-esters of long chain alcohols, alkylethersulfonic acids, like the alkylsulfuric acids; alkanesulfonic acids, olefinsulfonic acids or alkylbenzenesulfonates, preferably linear alkylbenzenesulfonates are also useful anionic surfactants. Alkanesulfonic acids can contain the sulfonic acid group terminally bound (primary alkanesulfonic acids) or along the C chain (secondary alkanesulfonic acids). Fatty alkyl sulfates, such as sodium octyl, decyl, lauryl, tetradecyl, hexadecyl, heptadecyl, or octadecyl sulfate; and salts of alkarylsulfonic acids, such as sodium octylbenzene sulfonates, are preferred.

Other useful anionic surfactants are those of the general formula R(OCH₂CH₂)ₙOSO₃M, wherein R is a C₁₀ to C₁₈ alkyl group, n is 1 to 3 and M is sodium; and salts of dialkyl sulfosuccinic acids, such as sodium dioctyl sulfosuccinate.

A preferred anionic surfactant is sodium lauryl sulfate.

Useful nonionic surfactants are alkoxylated, advantageously ethoxylated, especially primary alcohols with preferably 8 to 18 carbon atoms and an average of 1 to 12 mols ethylene oxide (EO) per mol alcohol, wherein the alcohol radical may be linear or preferably branched in 2-position with methyl, or may contain linear and methyl-branched radicals in a mixture, as customarily occurs in oxoalcohol radicals. In particular, however, alcohol ethoxylates with linear radicals made from alcohols of native origin with 12 to 18 carbon atoms, for example from coconut, palm, tallow fatty, or oleyl alcohols are preferred, and an average of 2 to 8 EO per mol alcohol. Preferred ethoxylated alcohols include, for example, C₁₂₋₁₄-alcohols with 3 EO or 4 EO, C₉₋₁₁-alcohols with 7 EO, C₁₃₋₁₅-alcohols with 3 EO, 5 EO, 7 EO or 8 EO, C₁₂₋₁₈-alcohols with 3 EO, 5 EO or 7 EO and mixtures of these, such as mixtures of C₁₂₋₁₄-alcohols with 3 EO and C₁₂₋₁₈-alcohols with 5 EO. The indicated degrees of ethoxylation represent statistical mean values that may be an integer or a fraction for a specific product. In addition to these nonionic surfactants, fatty alcohols with more than 12 EO may also be used. Examples are tallow fatty alcohols with 14 EO, 25 EO, 30 EO or 40 EO. Other preferred nonionic surfactants are ethoxylated reaction products of C₈₋₂₂-fatty alcohols, preferably C₁₂₋₂₀-fatty alcohols, and especially C₁₄₋₁₈-fatty alcohols with 1 to 30 mols ethylene oxide, preferably 2 to 20 mols ethylene oxide, and especially 5 to 10 mols ethylene oxide.

An additional class of preferably used nonionic surfactants is alkoxylated, preferably ethoxylated or ethoxylated and propoxylated fatty acid alkylesters, preferably with 1 to 4 carbon atoms in the alkyl chain, especially fatty acid methyl esters.

An additional class of useful nonionic surfactants is the alkylpolyglycosides (APG). Preferred alkylpolyglycosides have the general formula RO(G)_{z}, in which R represents a linear or branched, especially methyl-branched in 2-position, saturated or unsaturated aliphatic radical with 8 to 22, preferably 12 to 18 C-atoms and G is the symbol that represents a glycose unit with 5 or 6 C-atoms, preferably glucose. The glycosidation degree z here is from 1.0 to 4.0, preferably from 1.0 to 2.0, and especially from 1.1 to 1.5. Preferably used are linear alkylpolyglucosides, thus alkylpolyglycosides in which the polyglycol radical is a glucose radical and the alkyl radical is an n-alkyl radical.

An additional class of suitable nonionic surfactants are polyhydroxy fatty acid amides of Formula R—CO—N(R¹)-[Z], wherein R—CO represents an aliphatic acyl radical with 6 to 22 carbon atoms, R¹ represents hydrogen, an alkyl or hydroxyalkyl radical with 1 to 4 carbon atoms and [Z] represents a linear or branched polyhydroxyalkyl radical with 3 to 10 carbon atoms and 3 to 10 hydroxyl groups.

Useful cationic surfactants have cationic hydrophobic residues and counter-cations, such as chloride, sulfate, or acetate. Examples include tetraalkyl ammonium chlorides, aryl trialkyl ammonium chlorides, tetraalkyl ammonium bromides, aryl trialkyl ammonium bromides or N-alkylpyridinium chloride.

Amphoteric surfactants have zwitterionic hydrophilic groups. Examples thereof include aminocarboxylic acids, betaines, and sulfobetaines.

Generally a physiologically acceptable surfactant is used in the process of the present invention. Preferably the fluid composition which is used in step a) of the process of the present invention comprises a benzalkonium chloride (alkyl benzyl dimethylammonium chloride, CAS Registration number [8001-54-5]); cetrimide (hexadecyltrimethyl ammonium bromide, CAS Registration number [8044-71-1]); a glyceryl monooleate; a glyceryl monostearate; a glyceryl palmitostearate, CAS Registration number [8067-32-1]; a poloxamer (a polyethylene glycol, CAS Registration number [9003-11-6]); a polyoxyethylene alkyl ether, a polyoxyethylene castor oil derivative, a polyoxyethylene sorbitane fatty acid ester, such as a poly(oxyethylene)-sorbitane monooleate; a polyoxyethylene stearate, a sorbitane fatty acid ester, such as a sorbitane monooleate; and sodium lauryl sulfate, or a combination of two or more of the listed surfactants.

Fluid compositions used in step a) of the present invention which comprise the above-mentioned surfactants have a low viscosity. Such fluid compositions provide considerably advantages because they can be handled and processed to a foam easily upon contact with a gas. Furthermore, the produced foams break easily upon standing to recover the original fluid form, which is very advantageous for re-use, recycling or disposal of the fluid composition.

The fluid composition used in step a) of the process of the present invention may comprise a polymeric compound. However, in a preferred embodiment of the present invention the fluid composition used in step a) does not comprise a significant amount of a polymeric compounds. More preferably, the fluid composition used in step a) does not comprise a significant amount of a polymeric compound having a weight average of more than 9000. Even more preferably, the fluid composition does not comprise a significant amount of a polymeric compound having a weight average of more than 5000. Most preferably, the fluid composition does not comprise a significant amount of a polymeric compound having a weight average of more than 2000. The weight average molecular weight can be determined by light scattering according to the Standard Test Method ASTM D-4001-93 (1999).

The fluid composition used in step a) of the process of the present invention also comprises a liquid diluent in an amount indicated further above. The term “liquid diluent” means a diluent that is liquid at normal pressure and 25° C. The liquid diluent preferably is a monomeric compound or an oligomeric compound with a molecular weight of up to 500, preferably up to 300, most preferably up to 100. In case of oligomeric compounds the stated molecular weight is the weight average molecular weight. Useful organic liquids are alcohols, preferably monofunctional alcohols, such as methanol, ethanol or isopropyl or oils, such as paraffin oils, animal oils or vegetable oils. Most preferably, an aqueous liquid diluent is used, such a saline solution or water.

The fluid composition may comprise one or more additional components such as therapeutic agents, fillers, pigments, flavors or plasticizers. If present, their total amount is generally up to 75 percent, preferably up to 50 percent, more preferably up to 25 percent, based on the total weight of the fluid composition.

The fluid composition is contacted with a gas, such as oxygen, nitrogen, carbon dioxide or, preferably, air to produce a foam. Preferably a water-based air foam is produced. The term “air foam” is used in its industry-accepted sense to mean a foam made by physically mixing air into a fluid, and thus the term is distinct from chemical or carbon dioxide foam or halocarbon blown foam. The foam can be produced in a known manner by mechanically or physically entraining or dispersing the gas in the fluid composition, for example by pumping the fluid composition to air-aspirating, foam producing equipment. The gas and the fluid composition are generally contacted at such amounts to produce a foam with an overrun of 50 to 10,000 percent, preferably from 80 to 2,000 percent, more preferably from 100 to 1,500 percent. The overrun is measured at 25° C. and atmospheric pressure and defined below as
Overrun(%)=[(volume foam−volume fluid)/volume fluid]×100.

A two-phase foam may be composed of an aqueous phase and a gaseous phase or a non-aqueous liquid phase and a gaseous phase. A three-phase foam may comprise, in addition to aqueous and gaseous phases, insoluble solids or immiscible liquids. Such three-phase foams can also comprise dissolved solids in the aqueous or immiscible liquid phase or in both liquid phases. Four-phase foams may comprise, in addition to aqueous and gaseous phases, immiscible liquids and insoluble solids. In all foams, any immiscible liquid phase may be present as an oil-in-water or water-in-oil emulsion or as a simple dispersion.

In step b) of the process of the present invention the foam produced in step a) is contacted with solid particles of an average size of less than 2500 micrometers. The average size of the solid particles is determined according to ASTM D-502-89 (2003), the Standard Test Method (for soaps and other detergents) for particle size determination by sieving. The term “average size of the solid particles” as used herein means the weight average particle size. The foam can be contacted with a wide variety of solid particles of any shape, such as spherical, elliptic, or fibrous.

According to a preferred embodiment of the invention the solid particles are in the shape of a powder of an average particle size of less than 1000 micrometers, preferably less than 750 micrometers, most preferably less than 500 micrometers. Any powder is useful which traditionally has been coated or agglomerated with a liquid in the medical and veterinary science, such as ingredients of pharmaceutical granules or tablets. Exemplary thereof are pharmaceutical excipients, such as lactose, dicalcium phosphate, microcrystalline cellulose, sugars, minerals, cellulose powder, cellulose fibers, disintegrants, binders, lubricants, colorants, flavorants or combinations thereof or therapeutic agents. The foam can be contacted with one or more compounds in powder form.

The weight ratio between the foam and the solid particles is generally from 1:100 to 1:0.01, preferably from 1:20 to 1:0.2, more preferably from 1:10 to 1:0.5, most preferably from 1:5 to 1:1. Generally the foam and the solid particles are contacted in such ratios that the weight of the above-mentioned surfactant i) is from 0.001 to 15, preferably from 0.02 to 15, more preferably from 0.1 to 10, most preferably from 0.15 to 8 percent, based on the weight of the solid particles.

According to another preferred embodiment of the present invention the solid particles are in the shape of fibers. It has been found that the process of the present invention is very effective in the production of agglomerates of particles which are typically produced in fibrous shape, such as cellulose ethers or cellulose esters. By providing an effective process for agglomerating particles in fibrous shape, significant reductions of the dust level of the fibrous material can be achieved.

Advantageously, step b) is conducted in a mixing device, such as a high shear mixing device, a low shear mixing device, a fluidized bed granulator, a roller compactor or a spray dryer. Usually the solid particles are added to the mixing device before it set in operation, but it can also be added later. The contact of the foam with solid particles can be carried out in various ways.

According to one embodiment of the process step b) the mixing device is set into operation after the solid particles and the foam have been fed to the mixing device. Preferably, the solid particles are fed to the mixing device, foam is placed on top of the solid particles and the mixing device is subsequently set into operation. Advantageously, 50 percent or more, more preferably 80 percent or more, most preferably 90 percent or more of the total amount of foam used in step b) is placed on top of the solid particles before the mixing device is set into operation. This embodiment of the process of the present invention is advantageous because it prevents dust emission during the dispersion step b).

According to yet another embodiment of the process step b) the solid particles are fed into a mixing device and foam is added continuously or in portions to the mixing device while the mixing device is in operation.

According to yet another embodiment of the process step b), a part of the foam is added to the solid particles before the mixing device is set in operation and a part of the foam is added continuously or in portions to the solid particles while the mixing device is in operation.

According to the process of the present invention a surprisingly homogeneous dispersion of the surfactant and optional other foam components in the solid particles is achieved. Moreover, a simple device can be used for applying the foam to the solid particles, such as a simple tube. Expensive and complex atomizing devices that are commonly used for spraying fine droplets of liquids on solid particles are not necessary. The dispersion of the foam in the solid particles is achieved at a rate which is comparable to or even faster than the dispersion of a corresponding liquid spray in the solid particles. The process of the present invention is also useful for dispersing poorly water-soluble compounds, such as poorly water-soluble therapeutic agents, in the solid particles.

Depending on the type of surfactant and optional other components in the foam or in the solid particles, the components of the foam coat solid particles with or without agglomeration of the solid particles. If neither the fluid composition comprises a binder for the solid particles nor the mass of solid particles comprises an additive that acts as a binder for the solid particles upon contact with the foam, the particles are at least partially coated with the foam but usually no agglomeration takes place. This method is particularly useful for coating powders, such as pharmaceutical excipients, with therapeutic agents, colorants or other materials in case no agglomeration of the solid particles is desired.

The process of the present invention is particularly useful for agglomerating solid particles and for producing a granular material. If the fluid composition comprises a binder for the solid particles and/or the mass of solid particles comprises an additive that acts as a binder for the solid particles upon contact with the foam, the particles agglomerate upon contacting the foam with the solid particles. In a preferred embodiment of the invention the mass of solid particles comprises a polymeric binder, for example gum arabic, xanthan gum, gum karaya, gum tragacanth, gum ghatti, guar gum, exudate gums, seaweed gums, seed gums, microbial gums, carrageenan, dextran, gelatin, alginates, pectins, starches, polysaccharides, such as cellulose ethers or cellulose esters, starch derivatives, guar derivatives or xanthan derivatives. Useful starch derivatives are for example starch ethers, such as hydroxypropyl starch or carboxymethyl starch. Exemplary of guar derivatives are for example carboxymethyl guar, hydroxypropyl guar, carboxymethyl hydroxypropyl guar or cationized guar. Other examples of useful polymers are homo- or copolymers of ethylene imine, an unsaturated acid, such as acrylic acid or a salt thereof, an unsaturated amide, such as acrylamide, a vinyl polymer, such as vinylalcohol, a vinyl ester, such as vinylacetate, vinylpyrrolidone, vinyloxazolidone, vinylmethyloxazolidone, ethylene sulfonic acid, vinylamine, vinylpyrridine, an alkylglycol, a polyalkylene oxide, such as polyethylene oxide, or an oxyethylene alkylether. Such polymeric compounds are described in Patent Application WO 03/020244, the teaching of which is incorporated herein by reference. Preferred polymeric binders are cellulose ethers, particularly water-soluble cellulose ethers, such as methylcelluloses, hydroxypropyl methylcelluloses or hydroxypropyl celluloses, polyvinylpyrrolidones or carbomers, carbomers being polymers of an acrylic acid cross-linked with a polyfunctional compound.

The produced granular material can be subjected to one or more known compounding steps, such as drying, grinding, for example wet-milling or dry-milling, sieving, mixing with optional additional ingredients, for example lubricants, pressing into tablets or combinations thereof. The drying step can be carried out prior to or after the grinding step. Preferably, the produced granular material is dried and processed to a material of an average particle size of from 10 to 10,000 micrometers, preferably from 10 to 5,000 micrometers, determined according to ASTM D-502-89 (2003). The term “average size of the solid particles” as used herein means the weight average particle size.

A therapeutic agent is comprised in the fluid composition used in step a) of the process of the present invention or in the mass of solid particles used in step b) of the process of the present invention or in both. A wide variety of therapeutic agents that are useful in the medical or veterinary science can be used. The process of the present invention is particularly useful for preparing a pharmaceutical composition with a controlled drug release or an immediate drug release. Therapeutic agents which are useful in the process of the present invention include, but are not limited to, drugs acting on the central nervous system, drugs acting on the gastrointestinal tract, drugs acting on the cardiovascular system; antibiotic drugs; vitamins; vaccines; nutrients; nutritional supplements; drugs for analgesia; drugs for erectile dysfunction; hormones such as insulin, calcitonin or parathyroid hormone; nicotine for smoking cessation; antitussive agents; anaesthesics; anticonvulsants; sedatives; drugs for sleep induction, antimycotic drugs, such as ciclopirox olamine; anti-inflammatory drugs, such as diclofenac, benzidamine, piroxicam, tiaprofen, ketoprofen or tetridamine; corticosteroids, such as hydrocortisone; immunomodulators, such as Pidotimod; mucosecretolytic agents, such as sobrerol or carboxymethylcysteine, wound healing agents, vegetal extracts, amino acids, or xanthines, such as cyclopropylmethylxantine. Preferred drugs are Diphenhydramine HCl, Hydrochlorothiazide, Fluoxetine, Hydrocodone/APAP, Metformin, Albuterol Aerosol, Ranitidine HCl, Alprazolam, Atenolol, Lorazepam, Cephalexin, Propoxyphene-N/APAP, Enalapril, Buspirone HCl, Tamoxifen, Lisinopril, combinations of such drugs, and drugs or drug combinations under the Trademarks Lipitor, Zocor, Prevacid, Prilosec, Procrit, Zyprexa, Epogen, Celebrex, Zoloft, Paxil, Norvasc, Neurontin, Nexium, Vioxx, Risperdal, Pravachol, Fosamax, Plavix, Oxycontin and Celexa. Surprisingly homogeneous tablets are achieved when particles are agglomerated according to the process of the present invention to produce a granular material and the granular material is pressed to tablets. This means that no substantial variation in product quality is observed from one tablet to the next. Surprisingly homogeneous tablets are even achieved when the therapeutic is only slightly soluble, very slightly soluble or practically insoluble in the liquid diluent, such as water, that is used to prepare the foam. Accordingly, the process of the present invention is not only useful for preparing granular material and tablets that comprise one or more therapeutic agents that are very soluble, freely soluble or soluble in the liquid diluent, such as water, that is used to prepare the foam but also for preparing granular material and tablets that comprise one or more therapeutic agents that are sparingly soluble, slightly soluble, very slightly soluble or practically insoluble in such liquid diluent. As used herein, “very soluble” means that less than 1 weight part of diluent is used to dissolve 1 weight part of therapeutic agent. “Freely soluble” or “soluble” respectively means that from 1 to 10 weight parts or from 10 to 30 weight parts respectively of diluent are used to dissolve 1 weight part of therapeutic agent. “Sparingly soluble” or “slightly soluble” respectively means that from 30 to 100 weight parts or from 100 to 1000 weight parts respectively of diluent are used to dissolve 1 weight part of therapeutic agent. “Very slightly soluble” or “practically insoluble or insoluble” respectively means that from 1000 to 10,000 weight parts or at least 10,000 weight parts respectively of diluent are used to dissolve 1 weight part of therapeutic agent.

The amount of the therapeutic agent comprised in the mass of solid particles mainly depends on the nature of the therapeutic agent and can vary over a very broad range.

Generally it ranges from about 0.005 to about 95 weight percent, based on the total weight of the solid particles after the fluid composition has been dispersed therein according to the process of the present invention.

Depending on the composition of the coated particles or the granular material produced according to the process of the present invention, the coated particles or the granular material can be used as-produced, for example as fillings for pharmaceutical capsules, or can be further processed to the desired product, for example it may be pressed to tablets.

The present invention is further illustrated by the following examples which should not be construed to limit the scope of the present invention. All parts and percentages are by weight unless otherwise indicated.

The compounds listed in Table 1 below are used in the examples. The alkyl and hydroxyalkyl substitutions of the cellulose ether in the examples below are measured and calculated according to ASTM D3876. The apparent viscosity of the cellulose ether in the examples below is measured and normalized to a 2 weight percent aqueous solution using an Ubbelohde viscometer at 20° C.

TABLE 1

| Designation | Description |
|---|---|
| FFL-316 | Fast flow lactose, commercially available from DMV International |
| | Pharma and Foremost Farms USA, is a powder component |
| Avicel PH102 | Microcrystalline cellulose, commercially available from FMC |
| | Corporation, is a powder component |
| K4MP CR | Hydroxypropyl methylcellulose with a methoxyl substitution of 19-24 |
| | percent, a hydroxypropoxyl substitution of 7-12 percent and a viscosity |
| | of about 4,000 mPa · s. It is commercially available from The Dow |
| | Chemical Company under the Trademark METHOCEL K4MP CR |
| DPH | Diphenhydramine HCl, an antihistamine |
| HCTZ | Hydrochlorothiazide (6-chloro-3,4-dihydro-2H-1,2,4-benzothiadiazine-7- |
| | sulfonamide 1,1-dioxide), a diuretic and antihypertensive drug |
| Tween 80 | Poly(oxyethylene)(20)-sorbitane monooleate |
| SLS | Sodium lauryl sulfate surfactant |

### EXAMPLES 2-5 AND 7-10 AND COMPARATIVE EXAMPLES 1A AND 6A

A) Granulation

Granular materials are prepared in a 10-L, Powerx high shear granulator (Glatt Air Techniques Inc.). Tables 2a and 2b list the ingredients of the powder composition that are used to produce tablets. All the ingredients minus the magnesium stearate (tablet lubricant) are charged into the granulator and pre-mixed for 1-minute using a main blade speed of 300 rpm and a side-chopper blade speed of 1500 rpm. The model drugs are chosen based on their aqueous solubility. Diphenhydramine HCl (DPH) is chosen to represent a freely water soluble model drug. 1 g of Diphenhydramine HCl dissolves in 1 mL water (Reference: The Merck Index-12th edition (1996), page 561, monograph 3367). Hydrochlorothiazide (HCTZ) is chosen to represent a water-insoluble model drug (Reference: The Merck Index—12th edition (1996), page 818, monograph 4822).

In each of the Examples 2-5 and 7-10 1200 g of the above-mentioned ingredients are granulated by applying a foam produced from an aqueous solution of the surfactant listed in Table 3 below at a rate of 100 g/minute for 430 seconds. The granules are tray-dried overnight at 110° F. (43° C.) in an oven until dry. The dried granules are then milled using a CoMil grinder (model 197S, Quadro Engineering) equipped with a round-hole, grater-type screen (2A-062G031-23139) and a rotating impeller (2A-1601-173) at a milling rate of 1000 rpm. Portions of each of the milled granules are retained for particle size distribution testing. The particle size distribution testing is conducted on a sieve shaker (RoTap model B, W.S. Tyler Co.). Table 4 shows the results of the particle size distribution testing.

Powder compositions of Examples 1A and 6A are not granulated but directly compressed into tablets.

B) Tablet Pressing

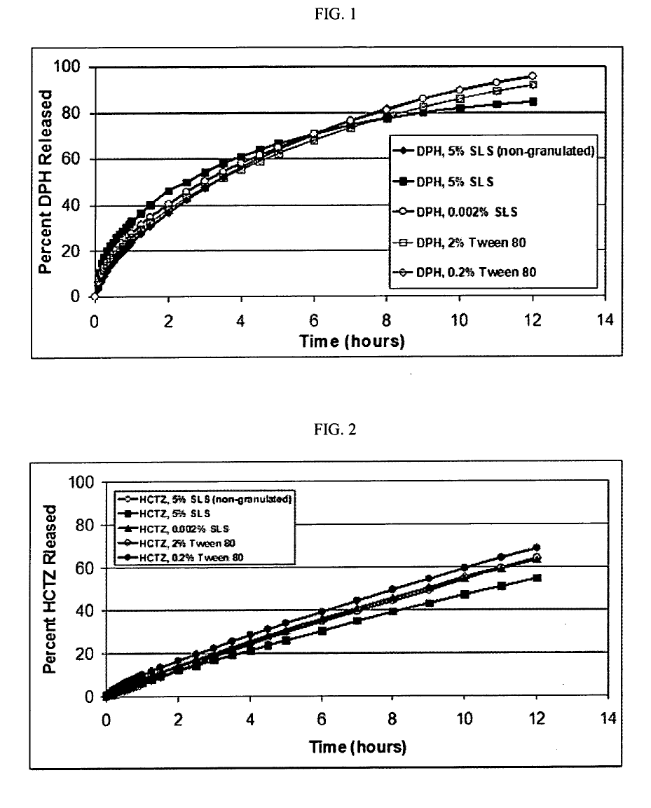

Magnesium stearate is mixed with the granulated material of Examples 2-5 and 7-10 or the non-granulated material of Examples 1A and 6A. Tablets are prepared on a Carver Press equipped with 10.3 mm, flat-faced, bevel-edged tooling and using a final compression force of 4500 lb (2025 kg). Drug dissolution testing is conducted on tablets representing each of the 10 tests. The results of the drug dissolution testing are shown in FIG. 1 and FIG. 2. FIG. 1 represents drug dissolution from prepared tablets containing the model drug DPH. FIG. 2 represents drug dissolution from prepared tablets containing the model drug HCTZ. A surprisingly homogeneous prepared tablet is achieved, even with the water-insoluble model drug hydrochlorothiazide, that means that no substantial variation in product quality is observed from one tablet to the next.

TABLE 2a

| Powder composition in Comparative Example 1A | |
|---|---|
| and Examples 2-5 | Weight percent |
| Diphenhydramine HCl (DPH) | 9 |
| METHOCEL K4MP CR | 30 |
| FFL-316 | 50.5 |
| Avicel PH-102 | 10 |
| Magnesium stearate | 0.5 |

TABLE 2b

| Powder composition in Comparative Example 7A | |
|---|---|
| and Examples 8-11 | Weight percent |
| Hydrochlorothiazide (HCTZ) | 2.4 |
| METHOCEL K4MP CR | 30 |
| FFL-316 | 57.1 |
| Avicel PH-102 | 10 |
| Magnesium stearate | 0.5 |

TABLE 3

| | | | | | Fluid Composition | |
|---|---|---|---|---|---|---|
| | Powder Components | | | | for foaming | |
| | K4MP | FLL-316 | Avicel PH- | Drug type/ | | Tween 80 |
| Example | (%)¹ | (%)¹ | 102 (%)¹ | level (%)¹ | SLS (%)² | (%)² |
| 1A | 30 | 50.5 | 10 | DHP/9 | — | — |
| 2 | 30 | 50.5 | 10 | DHP/9 | 5 | — |
| 3 | 30 | 50.5 | 10 | DHP/9 | 0.002 | — |
| 4 | 30 | 50.5 | 10 | DHP/9 | — | 2 |
| 5 | 30 | 50.5 | 10 | DHP/9 | — | 0.2 |
| 6A | 30 | 57.1 | 10 | HCTZ/2.4 | — | — |
| 7 | 30 | 57.1 | 10 | HCTZ/2.4 | 5 | — |
| 8 | 30 | 57.1 | 10 | HCTZ/2.4 | 0.002 | — |
| 9 | 30 | 57.1 | 10 | HCTZ/2.4 | — | 2 |
| 10 | 30 | 57.1 | 10 | HCTZ/2.4 | — | 0.2 |

¹weight percentage, based on total weight of powder
²weight percentage, based on total weight of fluid composition

TABLE 4

| | | Percent of granulation retained on each screen mesh | | | | | | |
|---|---|---|---|---|---|---|---|---|
| | | 20- | 40- | 60- | 80- | 100- | 140- | |
| (Comparative) Example | | mesh | mesh | mesh | mesh | mesh | mesh | Pan |
| 1A | DPH (non-granulated) | 0.1% | 1.7% | 2.8% | 6.3% | 10.9% | 24.2% | 51.9% |
| 2 | DPH, 5% SLS | 11.5% | 19.3% | 20.6% | 15.8% | 8.2% | 6.5% | 18.0% |
| 3 | DPH, 0.002% SLS | 55.5% | 28.9% | 6.1% | 2.3% | 1.0% | 1.0% | 4.8% |
| 4 | DPH, 2% Tween 80 | 40.9% | 33.1% | 10.9% | 4.0% | 1.5% | 1.4% | 7.8% |
| 5 | DPH, 0.2% Tween 80 | 42.8% | 31.4% | 9.5% | 4.2% | 1.9% | 1.8% | 7.8% |
| 6A | HCTZ (non-granulated) | 0.0% | 0.1% | 0.5% | 5.1% | 11.4% | 25.7% | 54.7% |
| 7 | HCTZ, 5% SLS | 55.5% | 28.9% | 6.1% | 2.3% | 7.8% | 1.0% | 4.8% |
| 8 | HCTZ, 0.002% SLS | 40.9% | 33.1% | 10.9% | 4.0% | 1.5% | 1.4% | 7.8% |
| 9 | HCTZ, 2% Tween 80 | 11.5% | 19.3% | 20.6% | 15.8% | 8.2% | 6.5% | 18.0% |
| 10 | HCTZ, 0.2% Tween 80 | 55.5% | 28.9% | 6.1% | 2.3% | 1.0% | 1.0% | 4.8% |

1. A process for dispersing a fluid in a mass of solid particles comprising the steps of
a) contacting a gas with a fluid composition comprising i) from 0.001 to 30 weight percent of a surfactant having a weight average molecular weight of up to 30000 and ii) from 99.999 to 70 weight percent of a liquid diluent, based on the total weight of the surfactant i) and the liquid diluent ii), and
b) contacting the produced foam with solid particles of an average size of up to 2500 micrometers,
wherein a therapeutic agent is comprised in the fluid composition or in the mass of solid particles or both.

2. A process for dispersing a fluid in a mass of solid particles comprising the steps of
a) contacting a gas with a fluid composition comprising i) from 0.001 to 30 weight percent of a surfactant having a weight average molecular weight of up to 9000 and ii) from 99.999 to 70 weight percent of a liquid diluent, based on the total weight of the surfactant i) and the liquid diluent ii), and
b) contacting the produced foam with solid particles of an average size of up to 2500 micrometers,
wherein a therapeutic agent is comprised in the fluid composition or in the mass of solid particles or both.

3. The process of claim 1 or claim 2 wherein the surfactant is physiologically acceptable.

4. The process of any one of claims 1 to 3 wherein the fluid composition comprises one or more surfactants selected from the list consisting of benzalkonium chlorides, hexadecyltrimethyl ammonium bromide, glyceryl monooleates, glyceryl monostearates, glyceryl palmitostearates, poloxamers, polyoxyethylene alkyl ethers, polyoxyethylene castor oil derivatives, polyoxyethylene sorbitan fatty acid esters, polyoxyethylene stearates, sorbitan fatty acid esters, and sodium lauryl sulfate.

5. The process of any one of claims 1 to 4 wherein the fluid composition does not comprise a polymeric compound having a weight average molecular weight of more than 9,000.

6. The process of any one of claims 1 to 5 wherein the fluid composition comprises i) from 0.01 to 10 weight percent of the surfactant and ii) from 99.99 to 90 weight percent of the liquid diluent ii).

7. The process of any one of claims 1 to 6 wherein the foam is a water-based air foam.

8. The process of any one of claims 1 to 7 wherein step b) comprises contacting the foam with the solid particles and agglomerating the particles to produce a granular material.

9. A granular material producible by the process of claim 8.

10. A process for preparing tablets comprising the steps of producing a granular material according to the process of claim 8 and pressing the granular material to tablets.
